(19) United States
(12) Patent Application Publication
(10) Pub. No.: US 2008/0286827 A1
(43) Pub. Date: Nov. 20, 2008

(54) BIOMARKERS

(75) Inventors: Steve Stone, Salt Lake City, UT (US); Alexander Gutin, Salt Lake City, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 12/102,692

(22) Filed: Apr. 14, 2008

Related U.S. Application Data
(60) Provisional application No. 60/923,244, filed on Apr. 12, 2007.

(51) Int. Cl. C12Q 1/02

(52) U.S. Cl. 435/29

(57) ABSTRACT

The invention relates to biomarkers for use in diagnosing cancer and in classifying tumors.

### CROSS-REFERENCE TO RELATED APPLICATIONS

This application claims priority under 35 U.S.C. § 119(e) to U.S. provisional application Ser. No. 60/923,244, filed Apr. 12, 2007, which is hereby incorporated by reference in its entirety.

### FIELD OF THE INVENTION

The invention relates to methods for treating cancer.

### BACKGROUND OF THE INVENTION

Due to years of clinical and basic research, major advances are being made in treating a number of cancers. One understanding that has emerged from this research over the years is that certain cancers seem to be dependent on, or have overactive growth factor signaling. Many cancers are characterized by amplifications in growth factor receptors which lead to amplification of growth factor signaling. Examples of these cancers include, but are not limited to, glioma, breast cancer, prostate cancer, colorectal cancer, lung cancer, etc. The overactive signaling pathways result in the modulation of transcription of genes related to survival and apoptosis. Many breast cancers, for example, are characterized by an amplification of HER2, a member of the ErbB family of growth factor receptor proteins that is overexpressed on the surface of cells of many cancers. A very successful new cancer drug, Herceptin™ (trastuzumab), is an antibody that binds to HER2. Clinical trials have shown that treatment of metastatic HER2-positive breast cancer with Herceptin in addition to chemotherapy increased patient survival compared to chemotherapy alone. More recently, Herceptin was approved by the FDA as adjuvant treatment for early stage HER2-positive cancers, since it was found that one year of treatment with Herceptin in these patients reduced the risk of death or recurrence by about 50%.

Another targeted therapy recently approved to treat breast cancer patients is Lapatinib (Tykerb™). Lapatinib is a small molecule tyrosine kinase inhibitor that targets EGFR, another growth factor receptor that, along with HER2, is overexpressed on the surface of cells of many cancers.

### BRIEF SUMMARY OF THE INVENTION

The invention relates to the identification of biomarkers and targets in cancer. More specifically, the invention relates to a set of cancer biomarkers. The cancer biomarkers can be used in a number of applications, including, but not limited to, assessing risk of rapid or slow disease progression, response to therapeutic treatment, choice of therapeutic treatment, prognosis, and diagnosis.

It has been discovered that biomarkers corresponding to the genes listed in Table 1 are important in certain cancers. Some of these genes are differentially expressed (or altered) amongst certain types of cancers while others are differentially expressed (or altered) within the same type of cancer. Accordingly, the differentially expressed (or altered) genes listed in Table 1, their expressed protein products, and/or corresponding copy number changes can be used in molecular medicine applications and as targets for cancer therapy. As a result of the invention, the genes listed in Table 1, as well as their expressed protein products, can be analyzed in samples for cancer diagnosis and prognosis. Another use of the genes in Table 1 is for the selection of therapeutic treatments based on the status of the genes and expressed protein products in Table 1. The genes (and expressed protein products) listed in Table 1 can also now be used as a drug target for cancer therapeutics.

In one embodiment, the invention provides a set of cancer biomarkers. According to this embodiment, the biomarkers relate to genes, mRNAs, and proteins corresponding to the biomarkers as described in Table 1 in the Example. A biomarker can be a specific gene listed in Table 1, alternative splice variants of the gene, fragments of genomic DNA comprising the gene (or a fragment thereof), mRNA molecules corresponding to the gene (or fragments thereof), cDNA corresponding to the gene (or fragments thereof), protein corresponding to the gene (or fragments thereof), and the like. In one aspect of this embodiment, a cancer is classified by measuring at least 1, 2, 3, 4, 5, 6, 7, 8, 9, 10, 11, 12, 13, 14, 15, 16, 17, 18, 19, 20, 25, 30, or 35 of the biomarkers listed in Table 1. In one aspect of this embodiment, a cancer is classified by measuring a set of at least 1, 2, 3, 4, 5, 6, 7, 8, 9, 10, 11, 12, 13, 14, 15, 16, 17, 18, 19, 20, 25, 30, or 35 of the biomarkers listed in Table 1, and comparing the measured values to a reference (or control). The set of biomarkers can be assessed according to the invention by a variety of methods. Such methods of characterizing whether a cancer has a biomarker signature according to the invention, include, but are not limited to, DNA copy number or sequence analysis of one or more genomic regions having the genes as listed in Table 1, RNA sequence or expression analysis of one or more genes as listed in Table 1, and detection of proteins expressed from one or more genes as listed in Table 1. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the biomarkers listed in Table 1.

In one embodiment, the invention provides a set of DNA copy number or sequence biomarkers. According to this embodiment, the biomarkers relate to genomic DNA regions corresponding to the biomarkers as described in Table 1 in the Example. The biomarker, according to this embodiment, can be a genomic region, marker, loci, or the such, comprising a specific gene listed in Table 1. In one aspect of this embodiment, a cancer is classified by measuring at least 1, 2, 3, 4, 5, 6, 7, 8, 9, 10, 11, 12, 13, 14, 15, 16, 17, 18, 19, 20, 25, 30, or 35 genomic regions corresponding to the biomarkers listed in Table 1. In one aspect of this embodiment, a cancer is classified by measuring and/or sequencing a set of at least 1, 2, 3, 4, 5, 6, 7, 8, 9, 10, 11, 12, 13, 14, 15, 16, 17, 18, 19, 20, 25, 30, or 35 genomic regions corresponding to the biomarkers listed in Table 1, and comparing the measured values to a reference (or control). The set of biomarkers can be assessed according to the invention by a variety of methods. Such methods of characterizing whether a cancer has a biomarker signature according to the invention, includes, but is not limited to, DNA copy number or sequence analysis of one or more genomic regions comprising at least one of the genes as listed in Table 1. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the genomic regions comprising at least one of the genes listed in Table 1.

In one embodiment, the invention provides a set of mRNA biomarkers. According to this embodiment, the biomarkers relate to mRNAs corresponding to the biomarkers as described in Table 1 in the Example. The mRNA biomarkers, according to this embodiment, can be any transcripts or cDNAs (or fragments thereof) that correspond to one or more of the genes listed in Table 1. In one aspect of this embodiment, a cancer is classified by measuring and/or sequencing at least 1, 2, 3, 4, 5, 6, 7, 8, 9, 10, 11, 12, 13, 14, 15, 16, 17, 18, 19, 20, 25, 30, or 35 mRNA biomarkers corresponding to the genes listed in Table 1. In one aspect of this embodiment, a cancer is classified by measuring and/or sequencing a set of at least 1, 2, 3, 4, 5, 6, 7, 8, 9, 10, 11, 12, 13, 14, 15, 16, 17, 18, 19, 20, 25, 30, or 35 mRNAs corresponding to the biomarkers listed in Table 1, and comparing the measured values and/or sequences to a reference (or control). The set of mRNA biomarkers can be assessed according to the invention by a variety of methods capable of ascertaining the mRNA expression level and/or sequence of a particular gene. Such methods of characterizing whether a cancer has a biomarker signature according to the invention, include, but are not limited to, microarray based mRNA expression analysis or quantitative PCR analysis of one or more transcripts (of fragments thereof) corresponding to the genes as listed in Table 1. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the mRNAs (or fragments thereof) corresponding to the genes listed in Table 1.

In one embodiment, the invention provides a set of protein biomarkers. According to this embodiment, the biomarkers relate to proteins corresponding to the biomarkers as described in Table 1 in the Example. The protein biomarkers, according to this embodiment, can be any protein (or fragments thereof) that correspond to one or more of the genes listed in Table 1. In one aspect of this embodiment, a cancer is classified by measuring at least 1, 2, 3, 4, 5, 6, 7, 8, 9, 10, 11, 12, 13, 14, 15, 16, 17, 18, 19, 20, 25, 30, or 35 protein biomarkers corresponding to the genes listed in Table 1. In one aspect of this embodiment, a cancer is classified by measuring a set of at least 1, 2, 3, 4, 5, 6, 7, 8, 9, 10, 11, 12, 13, 14, 15, 16, 17, 18, 19, 20, 25, 30, or 35 proteins corresponding to the biomarkers listed in Table 1, and comparing the measured values to a reference (or control). The set of protein biomarkers can be assessed according to the invention by a variety of methods capable of ascertaining protein expression levels of a particular protein. Such methods include, but are not limited to, monoclonal or polyclonal antibody based detection (via IHC, ELISA, or other suitable method) of proteins expressed from the one or more genes from Table 1.

In one embodiment, the invention provides a composition comprising a cancer biomarker probe set consisting from 2-1,000,000, 2-500,000, 2-100,000, 2-10,000, 2-1000, 2-500, 2-100, 2-50, 2-45, 2-40, 2-35, 2-30, 2-25, 2-20, 2-15, 2-14, 2-13, 2-12, 2-11, 2-10, 2-9, 2-8, 2-7, 2-6 or 2-5 different probes, wherein at least 40%, 50%, 60%, 70%, 80%, or 90% or more of the different probes are capable of detecting one or more biomarkers corresponding to the genes as in Table 1; wherein the different probes in total selectively detect at least 2, 3, 4, 5, 6, 7, 8, 9, 10, 11, 12, 13, 14, 15, 16, 17, 18, 19, or 20 of the different biomarkers in Table 1). As the skilled artisan is aware, probes to DNA, mRNA, and/or protein can be employed in the methods of the invention to detect the biomarkers. Such probes are commercially available or can be made by an ordinary skilled artisan in view of the GeneID numbers given in Table 1.

In one embodiment, the invention provides a composition comprising a cancer biomarker probe set consisting from 2-1,000,000, 2-500,000, 2-100,000, 2-10,000, 2-1000, 2-500, 2-100, 2-50, 2-45, 2-40, 2-35, 2-30, 2-25, 2-20, 2-15, 2-14, 2-13, 2-12, 2-11, 2-10, 2-9, 2-8, 2-7, 2-6 or 2-5 different probes, wherein at least 40%, 50%, 60%, 70%, 80%, or 90% or more of the different probes are capable of detecting one or more biomarkers which are nucleic acids corresponding to the genes as in Table 1; wherein the different probes in total selectively hybridize (or bind) to at least 2, 3, 4, 5, 6, 7, 8, 9, 10, 11, 12, 13, 14, 15, 16, 17, 18, 19, or 20 of the nucleic acids corresponding to the biomarker genes in Table 1.

In one embodiment, the invention provides a composition comprising a cancer biomarker probe set consisting from 2-1,000,000, 2-500,000, 2-100,000, 2-10,000, 2-1000, 2-500, 2-100, 2-50, 2-45, 2-40, 2-35, 2-30, 2-25, 2-20, 2-15, 2-14, 2-13, 2-12, 2-11, 2-10, 2-9, 2-8, 2-7, 2-6 or 2-5 different probes, wherein at least 40%, 50%, 60%, 70%, 80%, or 90% or more of the different probes are capable of detecting one or more biomarkers which are proteins corresponding to the genes as in Table 1; wherein the different probes in total selectively bind to at least 2, 3, 4, 5, 6, 7, 8, 9, 10, 11, 12, 13, 14, 15, 16, 17, 18, 19, or 20 of the proteins (or fragments thereof) corresponding to the biomarker genes in Table 1.

The invention provides a method for classifying a cancer tumor or tissue comprising: (a) contacting a sample (e.g., prostate or breast cancer sample) obtained from a subject suspected of having a tumor (or cancer) with probes that, in total, selectively detect at least 2, 3, 4, 5, 6, 7, 8, 9, 10, 11, 12, 13, 14, 15, 16, 17, 18, 19, or 20 different biomarkers corresponding to the genes listed in Table 1; wherein the contacting occurs under conditions to promote selective hybridization or binding of the probes to the biomarkers present in the sample; (b) detecting formation of hybridization or binding complexes between the probes biomarker targets, wherein a number of such hybridization or binding complexes provides a measure of one or more biomarkers corresponding to those listed in Table 1; and (c) correlating an alteration in the one or more biomarkers according to a characteristic (e.g., prognosis or potential efficacy of a particular treatment).

The invention provides a method for classifying a cancer tumor or tissue comprising: (a) contacting a nucleic acid sample (e.g., prostate or breast cancer sample) obtained from a subject suspected of having a tumor (or cancer) with nucleic acid probes that, in total, selectively hybridize to at least 2, 3, 4, 5, 6, 7, 8, 9, 10, 11, 12, 13, 14, 15, 16, 17, 18, 19, or 20 of the different genes in Table 1; wherein the contacting occurs under conditions to promote selective hybridization of the nucleic acid probes to the nucleic acid targets (regions), or complements thereof, present in the nucleic acid sample; (b) detecting formation of hybridization complexes between the nucleic acid probes to the nucleic acid targets, or complements thereof, wherein a number of such hybridization complexes provides a measure of gene copy number of the one or more nucleic acids according to genes listed in Table 1; and (c) correlating an alteration in the level of one or more nucleic acids according to the genes in Table 1 to a cancer classification (or characteristic) relative to a control (e.g., prognosis or potential efficacy of a particular treatment).

In another embodiment, the present invention provides a method for classifying a tumor or tissue comprising:

(a) contacting a mRNA-derived nucleic acid sample obtained from a subject having cancer with nucleic acid probes that, in total, selectively hybridize to at least 2, 3, 4, 5, 6, 7, 8, 9, 10, 11, 12, 13, 14, 15, 16, 17, 18, 19, or 20 mRNAs, or complement thereof corresponding to the genes provided in Table 1; wherein the contacting occurs under conditions to allow selective hybridization of the nucleic acid probes to the nucleic acid targets, or complements thereof, present in the nucleic acid sample;

(b) detecting formation of hybridization complexes between the nucleic acid probes to the nucleic acid targets, or complements thereof, wherein a number of such hybridization complexes provides a measure of gene expression of the one or more nucleic acids corresponding to a nucleic acid to those listed in Table 1; and

(c) correlating an alteration in gene expression of the one or more nucleic acids expressed from genes in Table 1, relative to control with a cancer classification (or characteristic).

In another embodiment, the present invention provides a method for classifying a tumor or tissue comprising:

(a) contacting a protein sample obtained from a subject having a cancer with probes that, in total, selectively bind to at least 2, 3, 4, 5, 6, 7, 8, 9, 10, 11, 12, 13, 14, 15, 16, 17, 18, 19, or 20 proteins, corresponding to proteins expressed from the different genes in Table 1; wherein the contacting occurs under conditions to promote binding of the probes to proteins in the sample;

(b) detecting binding of the probes to the proteins in the sample, wherein a number of such protein:probe complexes provides a measure of expression of the one or more nucleic acids corresponding to a gene as in Table 1; and

(c) correlating an alteration in gene expression of the one or more nucleic acids expressed from genes in Table 1, relative to control with a cancer classification (or characteristic).

In some aspects of the invention, the expression level (e.g., protein or mRNA) or copy number, of the one or more genes from Table 1 is determined by an analytically appropriate method chosen from a binding assay, reverse transcription polymerase chain reaction (RT-PCR), quantitative PCR, Northern hybridization, microarray analysis, enzyme immunoassay (EIA), two-hybrid assay, blot assay, and sandwich assay.

In one specific embodiment, differential expression of a biomarker of the invention refers to an expression value that is more than 2, 3, 4, or 5 standard deviations greater or lower than the average value.

In another specific embodiment, alteration of the copy number of a biomarker refers to in the case of deletions, loss-of-heterozygosity and homozygous deletions, or in the case of amplification in normal diploid cells, copy numbers of greater than 2, 3, 4, 5, 6, 7, 8, 9, or 10 for a particular biomarker.

In one embodiment, the invention relates to methods for identifying a patient having a cancer that will respond, is likely to respond, or is more likely to respond to an agent targeting a growth factor signaling pathway.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The invention relates to the identification of biomarkers and targets in cancer. More specifically, the invention relates to a set of cancer biomarkers. The cancer biomarkers can be used in a number of applications, including, but not limited to, assessing risk of rapid or slow disease progression, response to therapeutic treatment, etc.

It has been discovered that biomarkers corresponding to the genes listed in Table 1 are important in certain cancers. Some of these genes are differentially expressed (or altered) amongst certain types of cancers while others are differentially expressed (or altered) within the same type of cancer. Accordingly, the differentially expressed (or altered) genes listed in Table 1, their expressed protein products, and/or corresponding copy number changes can be used in molecular medicine applications and as targets for cancer therapy. As a result of the invention, the genes listed in Table 1, as well as their expressed protein products, can be analyzed in samples for cancer diagnosis and prognosis. Another use of the genes in Table 1 is for the selection of therapeutic treatments based on the status of the genes and expressed protein products in Table 1. The genes (and expressed protein products) listed in Table 1 can also now be used as a drug target for cancer therapeutics.

TABLE 1
Biomarkers

| | | | Entrez |
|---|---|---|---|
| | | | GeneID |
| GENES | Sequence | Expression | No. |
| PCDGF/GP88 | | X | 2896 |
| EGFR | X | X | 1956 |
| HER2 | X | X | 2064 |
| MUC4 | | X | 4585 |
| IGF-IR | | X | 3480 |
| p27 (kip1) | | X | 1027 |
| Akt | | X | 207 |
| HER3 | | X | 2065 |
| HER4 | | X | 2066 |
| PTEN | X | X | 5728 |
| PIK3CA | X | X | 5290 |
| SHIP | | X | 3635 |
| Grb2 | | X | 2885 |
| Gab2 | | X | 9846 |
| PDK-1 (3-phosphoinositide | | X | 5170 |
| dependent protein kinase-1) | | | |
| TSC1 | | X | 7248 |
| TSC2 | | X | 7249 |
| mTOR | | X | 2475 |
| MIG-6 (ERBB receptor feedback | | X | 54206 |
| inhibitor 1) | | | |
| S6K | | X | 6198 |
| src | | X | 6714 |
| KRAS | X | X | 3845 |
| BRAF | X | X | 673 |
| MEK mitogen-activated protein | | X | 4214 |
| kinase kinase kinase 1 | | | |
| cMYC | X | X | 4609 |
| TOPO II topoisomerase (DNA) II | | X | 7153 |
| alpha 170 kDa | | | |
| FRAP1 | | X | 2475 |
| NRG1 | | X | 3084 |
| ESR1 | | X | 2099 |
| ESR2 | | X | 2100 |
| PGR | | X | 5241 |
| CDKN1B | | X | 1027 |
| MAP2K1 | | X | 5604 |
| NEDD4-1 | | X | 4734 |
| FOXO3A | | X | 2309 |
| PPP1R1B | | X | 84152 |
| PXN | | X | 5829 |
| ELA2 | | X | 1991 |
| CTNNB1 | | X | 1499 |
| AR | | X | 367 |
| EPHB2 | | X | 2048 |
| KLF6 | | X | 1316 |
| ANXA7 | | X | 310 |
| NKX3-1 | | X | 4824 |
| PITX2 | | X | 5308 |
| MKI67 | | X | 4288 |
| PHLPP | | X | 23239 |

In one embodiment, the invention provides a set of cancer biomarkers. According to this embodiment, the biomarkers relate to genes, mRNAs, and proteins corresponding to the biomarkers as described in Table 1 in the Example. A biomarker can be a specific gene listed in Table 1, alternative splice variants of the gene, fragments of genomic DNA comprising the gene (or a fragment thereof), mRNA molecules corresponding to the gene (or fragments thereof), cDNA corresponding to the gene (or fragments thereof), protein corresponding to the gene (or fragments thereof), and the like. In one aspect of this embodiment, a cancer is classified by measuring at least 1, 2, 3, 4, 5, 6, 7, 8, 9, 10, 11, 12, 13, 14, 15, 16, 17, 18, 19, 20, 25, 30, or 35 of the biomarkers listed in Table 1. In one aspect of this embodiment, a cancer is classified by measuring a set of at least 1, 2, 3, 4, 5, 6, 7, 8, 9, 10, 11, 12, 13, 14, 15, 16, 17, 18, 19, 20, 25, 30, or 35 of the biomarkers listed in Table 1, and comparing the measured values to a reference (or control). The set of biomarkers can be assessed according to the invention by a variety of methods. Such methods of characterizing whether a cancer has a biomarker signature according to the invention, include, but are not limited to, DNA copy number analysis of one or more genomic regions having the genes as listed in Table 1, RNA expression analysis of the one or more genes as listed in Table 1, and detection of proteins expressed from the one or more genes as listed in Table 1. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the biomarkers listed in Table 1.

In one embodiment, the invention provides a set of DNA copy number or sequence biomarkers. According to this embodiment, the biomarkers relate to genomic DNA regions corresponding to the biomarkers as described in Table 1 in the Example. The biomarker, according to this embodiment, can be a genomic region, marker, loci, or the such, comprising a specific gene listed in Table 1. In one aspect of this embodiment, a cancer is classified by measuring and/or sequencing at least 1, 2, 3, 4, 5, 6, 7, 8, 9, 10, 11, 12, 13, 14, 15, 16, 17, 18, 19, 20, 25, 30, or 35 genomic regions corresponding to the biomarkers listed in Table 1. In one aspect of this embodiment, a cancer is classified by measuring a set of at least 1, 2, 3, 4, 5, 6, 7, 8, 9, 10, 11, 12, 13, 14, 15, 16, 17, 18, 19, 20, 25, 30, or 35 genomic regions corresponding to the biomarkers listed in Table 1, and comparing the measured values to a reference (or control). The set of biomarkers can be assessed according to the invention by a variety of methods. Such methods of characterizing whether a cancer has a biomarker signature according to the invention, includes, but is not limited to, DNA copy number or sequence analysis of one or more genomic regions comprising at least one of genes as listed in Table 1. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the genomic regions comprising at least one of the genes listed in Table 1.

In one embodiment, the invention provides a set of mRNA biomarkers. According to this embodiment, the biomarkers relate to mRNAs corresponding to the biomarkers as described in Table 1 in the Example. The mRNA biomarkers, according to this embodiment, can be any transcripts or cDNAs (or fragments thereof) that correspond to one or more of the genes listed in Table 1. In one aspect of this embodiment, a cancer is classified by measuring and/or sequencing at least 1, 2, 3, 4, 5, 6, 7, 8, 9, 10, 11, 12, 13, 14, 15, 16, 17, 18, 19, 20, 25, 30, or 35 mRNA biomarkers corresponding to the genes listed in Table 1. In one aspect of this embodiment, a cancer is classified by measuring and/or sequencing a set of at least 1, 2, 3, 4, 5, 6, 7, 8, 9, 10, 11, 12, 13, 14, 15, 16, 17, 18, 19, 20, 25, 30, or 35 mRNAs corresponding to the biomarkers listed in Table 1, and comparing the measured values and/or sequences to a reference (or control). The set of mRNA biomarkers can be assessed according to the invention by a variety of methods capable of ascertaining the mRNA expression level and/or sequence of a particular gene. Such methods of characterizing whether a cancer has a biomarker signature according to the invention, include, but are not limited to, microarray based mRNA expression analysis or quantitative PCR analysis of one or more transcripts (of fragments thereof) corresponding to the genes as listed in Table 1. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the mRNAs (or fragments thereof) corresponding to the genes listed in Table 1.

In one embodiment, the invention provides a set of protein biomarkers. According to this embodiment, the biomarkers relate to proteins corresponding to the biomarkers as described in Table 1 in the Example. The protein biomarkers, according to this embodiment, can be any protein (or fragments thereof) that correspond to one or more of the genes listed in Table 1. In one aspect of this embodiment, a cancer is classified by measuring at least 1, 2, 3, 4, 5, 6, 7, 8, 9, 10, 11, 12, 13, 14, 15, 16, 17, 18, 19, 20, 25, 30, or 35 protein biomarkers corresponding to the genes listed in Table 1. In one aspect of this embodiment, a cancer is classified by measuring a set of at least 1, 2, 3, 4, 5, 6, 7, 8, 9, 10, 11, 12, 13, 14, 15, 16, 17, 18, 19, 20, 25, 30, or 35 proteins corresponding to the biomarkers listed in Table 1 as compared to the measured values to a reference (or control). The set of protein biomarkers can be assessed according to the invention by a variety of methods capable of ascertaining protein expression levels of a particular protein. Such methods include, but are not limited to, monoclonal or polyclonal antibody based detection (via IHC, ELISA, or other suitable method) of proteins expressed from the one or more genes from Table 1.

In one embodiment, the invention provides a composition comprising a cancer biomarker probe set consisting from 2-1,000,000, 2-500,000, 2-100,000, 2-10,000, 2-1000, 2-500, 2-100, 2-50, 2-45, 2-40, 2-35, 2-30, 2-25, 2-20, 2-15, 2-14, 2-13, 2-12, 2-11, 2-10, 2-9, 2-8, 2-7, 2-6 or 2-5 different probes, wherein at least 40%, 50%, 60%, 70%, 80%, or 90% or more of the different probes are capable of detecting one or more biomarkers corresponding to the genes as in Table 1; wherein the different probes in total selectively detect at least 2, 3, 4, 5, 6, 7, 8, 9, 10, 11, 12, 13, 14, 15, 16, 17, 18, 19, or 20 of the different biomarkers in Table 1). As the skilled artisan is aware, probes to DNA, mRNA, and/or protein can be employed in the methods of the invention to detect the biomarkers

In one embodiment, the invention provides a composition comprising a cancer biomarker probe set consisting from 2-1,000,000, 2-500,000, 2-100,000, 2-10,000, 2-1000, 2-500, 2-100, 2-50, 2-45, 2-40, 2-35, 2-30, 2-25, 2-20, 2-15, 2-14, 2-13, 2-12, 2-11, 2-10, 2-9, 2-8, 2-7, 2-6 or 2-5 different probes, wherein at least 40%, 50%, 60%, 70%, 80%, or 90% or more of the different probes are capable of detecting one or more biomarkers which are nucleic acids corresponding to the genes as in Table 1; wherein the different probes in total selectively hybridize (or bind) to at least 2, 3, 4, 5, 6, 7, 8, 9, 10, 11, 12, 13, 14, 15, 16, 17, 18, 19, or 20 of the nucleic acids corresponding to the biomarker genes in Table 1.

In one embodiment, the invention provides a composition comprising a cancer biomarker probe set consisting from 2-1,000,000, 2-500,000, 2-100,000, 2-10,000, 2-1000, 2-500, 2-100, 2-50, 2-45, 2-40, 2-35, 2-30, 2-25, 2-20, 2-15, 2-14, 2-13, 2-12, 2-11, 2-10, 2-9, 2-8, 2-7, 2-6 or 2-5 different probes, wherein at least 40%, 50%, 60%, 70%, 80%, or 90% or more of the different probes are capable of detecting one or more biomarkers which are proteins corresponding to the genes as in Table 1; wherein the different probes in total selectively bind to at least 2, 3, 4, 5, 6, 7, 8, 9, 10, 11, 12, 13, 14, 15, 16, 17, 18, 19, or 20 of the proteins (or fragments thereof) corresponding to the biomarker genes in Table 1.

The invention provides a method for classifying a cancer tumor or tissue comprising: (a) contacting a sample (e.g., prostate or breast cancer sample) obtained from a subject suspected of having a tumor (or cancer) with probes that, in total, selectively detect at least 2, 3, 4, 5, 6, 7, 8, 9, 10, 11, 12, 13, 14, 15, 16, 17, 18, 19, or 20 different biomarkers corresponding to the genes listed in Table 1; wherein the contacting occurs under conditions to promote selective hybridization or binding of the probes to the biomarkers present in the sample; (b) detecting formation of hybridization or binding complexes between the probes biomarker targets, wherein a number of such hybridization or binding complexes provides a measure of one or more biomarkers corresponding to those listed in Table 1; and (c) correlating an alteration in the one or more biomarkers according to a characteristic (e.g., prognosis or potential efficacy of a particular treatment).

The invention provides a method for classifying a cancer tumor or tissue comprising: (a) contacting a nucleic acid sample (e.g., prostate or breast cancer sample) obtained from a subject suspected of having a tumor (or cancer) with nucleic acid probes that, in total, selectively hybridize to at least 2, 3, 4, 5, 6, 7, 8, 9, 10, 11, 12, 13, 14, 15, 16, 17, 18, 19, or 20 of the different genes in Table 1; wherein the contacting occurs under conditions to promote selective hybridization of the nucleic acid probes to the nucleic acid targets (regions), or complements thereof, present in the nucleic acid sample; (b) detecting formation of hybridization complexes between the nucleic acid probes to the nucleic acid targets, or complements thereof, wherein a number of such hybridization complexes provides a measure of gene copy number of the one or more nucleic acids according to genes listed in Table 1; and (c) correlating an alteration in the level of one or more nucleic acids according to the genes in Table 1 relative to a characteristic (e.g., prognosis or potential efficacy of a particular treatment).

In another embodiment, the present invention provides a method for classifying a tumor or tissue comprising:

(a) contacting a mRNA-derived nucleic acid sample obtained from a subject having cancer with nucleic acid probes that, in total, selectively hybridize to at least 2, 3, 4, 5, 6, 7, 8, 9, 10, 11, 12, 13, 14, 15, 16, 17, 18, 19, or 20 mRNAs, or complement thereof corresponding to the genes expressed in Table 1; wherein the contacting occurs under conditions to promote selective hybridization of the nucleic acid probes to the nucleic acid targets, or complements thereof, present in the nucleic acid sample;

(b) detecting formation of hybridization complexes between the nucleic acid probes to the nucleic acid targets, or complements thereof, wherein a number of such hybridization complexes provides a measure of gene expression of the one or more nucleic acids corresponding to a nucleic acid to those listed in Table 1; and

(c) correlating an alteration in gene expression of the one or more nucleic acids expressed from genes in Table 1, relative to control with a cancer classification.

In another embodiment, the present invention provides a method for classifying a tumor or tissue comprising:

(a) contacting a protein sample obtained from a subject having a cancer with probes that, in total, selectively bind to at least 2, 3, 4, 5, 6, 7, 8, 9, 10, 11, 12, 13, 14, 15, 16, 17, 18, 19, or 20 proteins, corresponding to proteins expressed from the different genes in Table 1; wherein the contacting occurs under conditions to promote binding of the probes to proteins in the sample;

(b) detecting binding of the probes to the proteins in the sample, wherein a number of such protein:probe complexes provides a measure of expression of the one or more nucleic acids corresponding to a gene as in Table 1; and

(c) correlating an alteration in gene expression of the one or more nucleic acids expressed from genes in Table 1, relative to control with a cancer classification.

The present invention provides novel compositions and methods for their use in classifying tumors and cancers. “Classifying” or “classification” according the methods of the invention, means to determine one or more features of the tumor (or cancer) or the prognosis of a patient from whom tissue sample is taken, including, but not limited to: (a) diagnosis of cancer; (b) metastatic potential, potential to metastasize to specific organs, risk of recurrence, or course of the tumor; (c) stage of the tumor; (d) patient prognosis in the absence of therapy treatment of the cancer; (e) prognosis of patient response to treatment (chemotherapy, radiation therapy, and/or surgery to excise tumor); (f) diagnosis of actual patient response to current and/or past treatment; (g) predicted optimal course of treatment for the patient; (h) prognosis for patient relapse after treatment; (i) patient life expectancy, etc.

Cancers (or suspected cancers) that may be so classified according to the invention include, but are not limited to: Hodgkin's disease, non-Hodgkin's lymphoma, acute lymphocytic leukemia, chronic lymphocytic leukemia, multiple myeloma, neuroblastoma, glioblastoma, breast cancer, ovarian cancer, lung cancer, Wilms' tumor, cervical cancer, testicular cancer, soft-tissue sarcoma, macroglobulinemia, bladder cancer, chronic granulocytic leukemia, brain cancer, malignant melanoma, small-cell lung cancer, stomach cancer, colon cancer, malignant pancreatic insulinoma, malignant carcinoid cancer, choriocancer, mycosis fungoides, head or neck cancer, osteogenic sarcoma, pancreatic cancer, acute granulocytic leukemia, hairy cell leukemia, neuroblastoma, rhabdomyosarcoma, Kaposi's sarcoma, genitourinary cancer, thyroid cancer, esophageal cancer, malignant hypercalcemia, cervical hyperplasia, renal cell cancer, endometrial cancer, polycythemia vera, essential thrombocytosis, adrenal cortex cancer, skin cancer, ovarian cancer, endometrial cancer, prostatic cancer, cancer of unknown origin, etc.

In some aspects of these embodiments, the biomarkers from the cancer cells, tumor, or tissue, are obtained from one or more tissues independently chosen from brain, lung, liver, spleen, kidney, lymph node, small intestine, pancreas, colon, stomach, breast, endometrial, prostate, testicle, ovary, skin, head and neck, esophagus, and bone marrow. The biomarkers can be in the form of genomic DNA, mRNA (or cDNA), or proteins.

In some aspects of the invention, the expression level (e.g., protein or mRNA) or copy number, of the one or more genes from Table 1 is determined by an analytically appropriate method chosen from a binding assay, reverse transcription polymerase chain reaction (RT-PCR), quantitative PCR, Northern hybridization, microarray analysis, enzyme immunoassay (EIA), two-hybrid assay, blot assay, and sandwich assay.

In one aspect, the invention provides a method comprising, obtaining a test sample from cells or tissue; determining the number of gene copies of one or more genes chosen from Table 1, per cell and comparing the number of gene copies per cell (for example, quantitatively and/or qualitatively) in the sample to a control sample or a known value, thereby determining whether one or more genes chosen from Table 1 are amplified or deleted in the test sample. Amplification of one or more amplified genes or deletion of one or more deleted genes chosen from Table 1 can indicate a cancer or a precancerous condition in the tissue, or can be used for prognosis or therapeutic decisions. In one aspect of this embodiment, the method involves identifying a patient in need of analysis of one or more genes chosen from Table 1 (e.g., a patient suspected of having a cancer in which the one or more amplified/deleted genes is amplified or deleted).

In another aspect, the present invention provides methods for diagnosing or predicting a cancer. The method of this aspect can comprise (1) obtaining a test sample from cells or tissue, (2) obtaining a control sample from cells or tissue that is normal, and (3) detecting or measuring in both the test sample and the control sample the level of one or more mRNA transcripts corresponding to one or more genes listed in Table 1. If the level of the one or more transcripts is higher in the test sample than that in the control sample, this indicates a cancer or a precancerous condition in the test sample cells or tissue. If the level of the one or more transcripts is lower in the test sample than that in the control sample, this indicates a cancer or a precancerous condition in the test sample cells or tissue. In another aspect the control sample may be obtained from a different individual or be a normalized value based on baseline data obtained from a population. In one aspect of this embodiment, the method involves identifying a patient in need of analysis of one or more genes from Table 1.

In yet another aspect, the invention provides a method comprising, obtaining a test sample from cells or tissue; detecting the number of DNA copies of one or more genes from Table 1 ((e.g., per cell) in the sample; and comparing the number of DNA copies detected (for example, quantitatively and/or qualitatively) in the sample to a control sample or a known value, thereby determining whether the one or more genes is amplified and/or deleted in the test sample. In one aspect of this embodiment, the method involves identifying a patient in need of analysis of one or more genes from Table 1.

In yet another aspect, the invention provides a method comprising (1) obtaining a test sample from cells or tissue; contacting the sample with an antibody to one or more expression products of one or more genes chosen from Table 1, and detecting in the test sample, the level of expression of one or more genes from Table 1, wherein an increased level or decreased level of the expression of one or more genes from Table 1 in the test sample, as compared to a control sample or a known value, indicates a precancerous or a cancerous condition in the cells or tissue. In another aspect, the control sample may be obtained from a different individual or be a normalized value based on baseline data obtained from a population. Alternatively, a given level of one or more genes from Table 1, representative of the cancer-free population, that has been previously established based on measurements from normal, cancer-free patients, can be used as a control. A control data point from a reference database, based on data obtained from control samples representative of a cancer-free population, also can be used as a control. In one aspect of this embodiment, the method involves identifying a patient in need of analysis of one or more genes from Table 1.

In some aspects of these embodiments, one or more genes that are examined for alterations are chosen from tumor suppressors or oncogenes. In a more specific aspect, one or more auxiliary genes are chosen from p53, PTEN, p16, c20orf133, TGF-β2, ctnna1, ctnnb1, KRAS, BRAF, and pik3ca. In a specific aspect, the DNA sequence of a nucleic acid corresponding to the one or more auxiliary genes is analyzed.

### DEFINITIONS

The terms “genetic variant” and “nucleotide variant” are used herein interchangeably to refer to changes or alterations to the reference human gene or cDNA sequence at a particular locus, including, but not limited to, nucleotide base deletions, insertions, inversions, and substitutions in the coding and non-coding regions. Deletions may be of a single nucleotide base, a portion or a region of the nucleotide sequence of the gene, or of the entire gene sequence. Insertions may be of one or more nucleotide bases. The “genetic variant” or “nucleotide variants” may occur in transcriptional regulatory regions, untranslated regions of mRNA, exons, introns, or exon/intron junctions. The “genetic variant” or “nucleotide variants” may or may not result in stop codons, frame shifts, deletions of amino acids, altered gene transcript splice forms or altered amino acid sequence.

The term “allele” or “gene allele” is used herein to refer generally to a naturally occurring gene having a reference sequence or a gene containing a specific nucleotide variant.

As used herein, “haplotype” is a combination of genetic (nucleotide) variants in a region of an mRNA or a genomic DNA on a chromosome found in an individual. Thus, a haplotype includes a number of genetically linked polymorphic variants which are typically inherited together as a unit.

As used herein, the term “amino acid variant” is used to refer to an amino acid change to a reference human protein sequence resulting from “genetic variants” or “nucleotide variants” to the reference human gene encoding the reference protein. The term “amino acid variant” is intended to encompass not only single amino acid substitutions, but also amino acid deletions, insertions, and other significant changes of amino acid sequence in the reference protein.

The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous. Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s).

The term “locus” refers to a specific position or site in a gene sequence or protein. Thus, there may be one or more contiguous nucleotides in a particular gene locus, or one or more amino acids at a particular locus in a polypeptide. Moreover, “locus” may also be used to refer to a particular position in a gene where one or more nucleotides have been deleted, inserted, or inverted.

As used herein, the terms “polypeptide,” “protein,” and “peptide” are used interchangeably to refer to an amino acid chain in which the amino acid residues are linked by covalent peptide bonds. The amino acid chain can be of any length of at least two amino acids, including full-length proteins. Unless otherwise specified, the terms “polypeptide,” “protein,” and “peptide” also encompass various modified forms thereof, including but not limited to glycosylated forms, phosphorylated forms, etc. The terms “primer”, “probe,” and “oligonucleotide” are used herein interchangeably to refer to a relatively short nucleic acid fragment or sequence. They can be DNA, RNA, or a hybrid thereof, or chemically modified analog or derivatives thereof. Typically, they are single-stranded. However, they can also be double-stranded having two complementing strands which can be separated apart by denaturation. Normally, they have a length of from about 8 nucleotides to about 200 nucleotides, preferably from about 12 nucleotides to about 100 nucleotides, and more preferably about 18 to about 50 nucleotides. They can be labeled with detectable markers or modified in any conventional manners for various molecular biological applications.

The term “isolated” when used in reference to nucleic acids (e.g., genomic DNAs, cDNAs, mRNAs, or fragments thereof) is intended to mean that a nucleic acid molecule is present in a form that is substantially separated from other naturally occurring nucleic acids that are normally associated with the molecule. Specifically, since a naturally existing chromosome (or a viral equivalent thereof) includes a long nucleic acid sequence, an “isolated nucleic acid” as used herein means a nucleic acid molecule having only a portion of the nucleic acid sequence in the chromosome but not one or more other portions present on the same chromosome. More specifically, an “isolated nucleic acid” typically includes no more than 25 kb naturally occurring nucleic acid sequences which immediately flank the nucleic acid in the naturally existing chromosome (or a viral equivalent thereof). However, it is noted that an “isolated nucleic acid” as used herein is distinct from a clone in a conventional library such as genomic DNA library and cDNA library in that the clone in a library is still in admixture with almost all the other nucleic acids of a chromosome or cell. Thus, an “isolated nucleic acid” as used herein also should be substantially separated from other naturally occurring nucleic acids that are on a different chromosome of the same organism. Specifically, an “isolated nucleic acid” means a composition in which the specified nucleic acid molecule is significantly enriched so as to constitute at least 10% of the total nucleic acids in the composition.

An “isolated nucleic acid” can be a hybrid nucleic acid having the specified nucleic acid molecule covalently linked to one or more nucleic acid molecules that are not the nucleic acids naturally flanking the specified nucleic acid. For example, an isolated nucleic acid can be in a vector. In addition, the specified nucleic acid may have a nucleotide sequence that is identical to a naturally occurring nucleic acid or a modified form or mutein thereof having one or more mutations such as nucleotide substitution, deletion/insertion, inversion, and the like.

An isolated nucleic acid can be prepared from a recombinant host cell (in which the nucleic acids have been recombinantly amplified and/or expressed), or can be a chemically synthesized nucleic acid having a naturally occurring nucleotide sequence or an artificially modified form thereof.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a composition or preparation in which the specified polypeptide molecule is significantly enriched so as to constitute at least 10% of the total protein content in the composition. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

The term “high stringency hybridization conditions,” when used in connection with nucleic acid hybridization, means hybridization conducted overnight at 42 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, means hybridization conducted overnight at 37 degrees C. in a solution containing 50% formamide, 5×SSC (750 mM NaCl, 75 mM sodium citrate), 50 mM sodium phosphate, pH 7.6, 5×Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

For the purpose of comparing two different nucleic acid or polypeptide sequences, one sequence (test sequence) may be described to be a specific “percentage identical to” another sequence (comparison sequence) in the present disclosure. In this respect, the percentage identity is determined by the algorithm of Karlin and Altschul, Proc. Natl. Acad. Sci. USA, 90:5873-5877 (1993), which is incorporated into various BLAST programs. Specifically, the percentage identity is determined by the “BLAST 2 Sequences” tool, which is available at NCBI's website. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a comparison sequence using BLAST 2.1.2., determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and comparison sequence is considered to be zero and the percent identity is calculated by adding the number of identical amino acids or nucleotides in the aligned regions and dividing that number by the length of the comparison sequence.

The Entrez GeneID numbers for the genes in Table 1 are provided merely as representative examples of a wild-type human sequence. These sequences are representative of one particular individual in the population of humans. Humans vary from one to another in their gene sequences. These variations are very minimal, sometimes occurring at a frequency of about 1 to 10 nucleotides per gene. Different forms of any particular gene exist within the human population. These different forms are called allelic variants. Allelic variants often do not change the amino acid sequence of the encoded protein; such variants are termed synonymous. Even if they do change the encoded amino acid (non-synonymous), the function of the protein is not typically affected. Such changes are evolutionarily or functionally neutral. When a human gene is referred to in the present application all allelic variants are intended to be encompassed by the term. The invention is not limited to this single allelic form of these genes or the proteins they encode.

### Gene Expression Profiling

In some aspects of the inventions, the biomarkers are assessed by gene expression profiling. In general, methods of gene expression profiling can be divided into two large groups: methods based on hybridization analysis of polynucleotides, and methods based on sequencing of polynucleotides. Commonly used methods known in the art for the quantification of mRNA expression in a sample include northern blotting and in situ hybridization (Parker & Barnes (1999) Methods in Molecular Biology 106:247-283); RNAse protection assays (Hod (1992) Biotechniques 13:852-854); and reverse transcription polymerase chain reaction (RT-PCR) (Weis et al. (1992) Trends in Genetics 8:263-264). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

### Reverse Transcriptase PCR (RT-PCR)

RT-PCR can be used to determine the mRNA levels of the biomarkers of the invention. RT-PCR can be used to compare mRNA levels of the biomarkers of the invention in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al. (1997) Current Protocols of Molecular Biology, John Wiley and Sons. Methods for RNA extraction from paraffin embedded tissues are disclosed, for example, in Rupp & Locker (1987) Lab Invest. 56:A67, and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Numerous RNA isolation kits are commercially available and can be used in the methods of the invention.

One of the first steps in gene expression profiling by RT-PCR is the reverse transcription of the RNA template into cDNA, followed by amplification in a PCR reaction. Commonly used reverse transcriptases include, but are not limited to, avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In one specific embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR. See, e.g. Held et al. (1996) Genome Research 6:986-994.

### Microarrays

The biomarkers of the invention can also be identified, confirmed, and/or measured using the microarray technique. Thus, the expression profile biomarkers can be measured in either fresh or paraffin-embedded tumor tissue, using microarray technology. In this method, polynucleotide sequences of interest are plated, or arrayed, on a microchip substrate. The arrayed sequences are then hybridized with specific DNA probes from cells or tissues of interest. As with the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. In one aspect, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al. (1996) Proc. Natl. Acad. Sci. USA 93(2):106-149). Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

### Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al. (1995) Science 270:484-487; and Velculescu et al. (1997) Cell 88:243-51.

### Gene Expression Analysis by Massively Parallel Signature Sequencing (MPSS)

This method, described by Brenner et al. (2000) Nature Biotechnology 18:630-634, is a sequencing approach that combines non-gel-based signature sequencing with in vitro cloning of millions of templates on separate microbeads. First, a microbead library of DNA templates is constructed by in vitro cloning. This is followed by the assembly of a planar array of the template-containing microbeads in a flow cell at a high density. The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

### DNA Copy Number Profiling

The invention is not intended to be limited by the specific method used to determine the DNA copy number profile of a particular sample. Any method capable of providing DNA copy number profiles can be used as along as the resolution is sufficient to identify the biomarkers of the invention. The skilled artisan is aware of and capable of using a number of different platforms for assessing whole genome copy number changes at a resolution sufficient to identify the copy number of the one or more biomarkers of the invention. Some of the platforms and techniques are described in the embodiments below.

In some aspects of these embodiments, the copy number profile analysis involves amplification of whole genome DNA by a whole genome amplification method. In a more specific aspect, the whole genome amplification method uses a strand displacing polymerase and random primers.

In some aspects of these embodiments, the copy number profile analysis involves hybridization of whole genome amplified DNA with a high density array. In a more specific aspect, the high density array has 5,000 or more different probes. In another specific aspect, the high density array has 5,000, 10,000, 20,000, 50,000, 100,000, 200,000, 300,000, 400,000, 500,000, 600,000, 700,000, 800,000, 900,000, or 1,000,000 or more different probes. In another specific aspect, each of the different probes on the array is an oligonucleotide having from about 15 to 200 bases in length. In another specific aspect, each of the different probes on the array is an oligonucleotide having from about 15 to 200, 15 to 150, 15 to 100, 15 to 75, 15 to 60, or 20 to 55 bases in length.

In many of the embodiment describe below, a microarray is employed to aid in determining the copy number profile for cells from a tumor. Microarrays typically comprise a plurality of oligomers (e.g., DNA or RNA polynucleotides or oligonucleotides, or other polymers), synthesized or deposited on a substrate (e.g., glass support) in an array pattern. The support-bound oligomers are “probes”, which function to hybridize or bind with a sample material (e.g., nucleic acids prepared or obtained from the tumor samples), in hybridization experiments. The reverse situation can also be applied: the sample can be bound to the microarray substrate and the oligomer probes are in solution for the hybridization. In use, the array surface is contacted with one or more targets under conditions that promote specific, high-affinity binding of the target to one or more of the probes. In some configurations, the sample nucleic acid is labeled with a detectable label, such as a fluorescent tag, so that the hybridized sample and probes are detectable with scanning equipment. DNA array technology offers the potential of using a multitude (e.g., hundreds of thousands) of different oligonucleotides to analyze DNA copy number profiles. In some embodiments, the substrates used for arrays are surface-derivatized glass or silica, or polymer membrane surfaces (see e.g., in Z. Guo, et al., Nucleic Acids Res, 22, 5456-65 (1994); U. Maskos, E. M. Southern, Nucleic Acids Res, 20, 1679-84 (1992), and E. M. Southern, et al., Nucleic Acids Res, 22, 1368-73 (1994), each incorporated by reference herein). Modification of surfaces of array substrates can be accomplished by many techniques. For example, siliceous or metal oxide surfaces can be derivatized with bifunctional silanes, i.e., silanes having a first functional group enabling covalent binding to the surface (e.g., Si-halogen or Si-alkoxy group, as in —SiCl₃ or —Si(OCH₃)₃, respectively) and a second functional group that can impart the desired chemical and/or physical modifications to the surface to covalently or non-covalently attach ligands and/or the polymers or monomers for the biological probe array. Silylated derivatizations and other surface derivatizations that are known in the art (see for example U.S. Pat. No. 5,624,711 to Sundberg, U.S. Pat. No. 5,266,222 to Willis, and U.S. Pat. No. 5,137,765 to Farnsworth, each incorporated by reference herein). Other processes for preparing arrays are described in U.S. Pat. No. 6,649,348, to Bass et. al., assigned to Agilent Corp., which disclose DNA arrays created by in situ synthesis methods.

Polymer array syntheses is also described extensively in the literature including in the following: WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, 5,412,087, 6,147,205, 6,262,216, 6,310,189, 5,889,165, and 5,959,098 in PCT Applications Nos. PCT/US99/00730 (International Publication No. WO 99/36760) and PCT/US01/04285 (International Publication No. WO 01/58593), which are all incorporated herein by reference in their entirety for all purposes.

Nucleic acid arrays that are useful in the present invention include, but are not limited to, those that are commercially available from Affymetrix (Santa Clara, Calif.) under the brand name GeneChip™. Example arrays are shown on the website at affymetrix.com. Another microarray supplier is illumina of San Diego, Calif. with example arrays shown on their website at illumina.com.

In some embodiments, the inventive methods provide for sample preparation. Depending on the microarray and experiment to be performed, sample nucleic acid can be prepared in a number of ways by methods known to the skilled artisan. In some aspects of the invention, prior to or concurrent with genotyping (analysis of copy number profiles), the sample may be amplified any number of mechanisms. The most common amplification procedure used involves PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. In some embodiments, the sample may be amplified on the array (e.g., U.S. Pat. No. 6,300,070 which is incorporated herein by reference)

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), 09/910,292 (U.S. Patent Application Publication 20030082543), and 10/013,598.

Methods for conducting polynucleotide hybridization assays are well developed in the art. Hybridization assay procedures and conditions used in the methods of the invention will vary depending on the application and are selected in accordance with the general binding methods known including those referred to in: Maniatis et al. Molecular Cloning: A Laboratory Manual (2.sup.nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The methods of the invention may also involve signal detection of hybridization between ligands in after (and/or during) hybridization. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

### Data and Analysis

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet as shown in U.S. Ser. Nos. 10/197,621, 10/063,559 (U.S. Publication Number 20020183936), 10/065,856, 10/065,868, 10/328,818, 10/328,872, 10/423,403, and 60/482,389.

### Methods for Analyzing Auxiliary Genes/Biomarkers

The present invention also provides a method for genotyping one or more auxiliary genes (and/or biomarkers in Table 1) by determining whether an individual has one or more nucleotide variants (or amino acid variants) in one or more of the auxiliary genes (or proteins). Genotyping one or more auxiliary genes according to the methods of the invention in some embodiments, can provide more evidence for determining therapy, diagnosis, and prognosis.

The auxiliary genes (and/or biomarkers in Table 1) of the invention can be analyzed by any method useful for determining alterations in nucleic acids or the proteins they encode. According to one embodiment, the ordinary skilled artisan can analyze the one or more auxiliary genes for mutations including deletion mutants, insertion mutants, frameshift mutants, nonsense mutants, missense mutant, and splice mutants.

Nucleic acid used for analysis of the one or more auxiliary genes (and/or biomarkers from Table 1) can be isolated from cells in the sample according to standard methodologies (Sambrook et al., 1989). The nucleic acid, for example, may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified. Depending on the format of the assay for analyzing the one or more auxiliary tumors suppressor genes, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects are known to occur in the auxiliary genes (and/or biomarkers of Table 1) of the invention. Thus, “alterations” should be read as including deletions, insertions, point mutations, and duplications. Point mutations result in stop codons, frameshift mutations or amino acid substitutions. Mutations in and outside the coding region of the one or more auxiliary genes may occur and can be analyzed according to the methods of the invention.

Similarly, a method for haplotyping one or more auxiliary genes is also provided. Haplotyping can be done by any methods known in the art. For example, only one copy of one or more auxiliary genes can be isolated from an individual and the nucleotide at each of the variant positions is determined. Alternatively, an allele specific PCR or a similar method can be used to amplify only one copy of the one or more auxiliary genes in an individual, and the SNPs at the variant positions of the present invention are determined. The Clark method known in the art can also be employed for haplotyping. A high throughput molecular haplotyping method is also disclosed in Tost et al., Nucleic Acids Res., 30(19):e96 (2002), which is incorporated herein by reference.

Thus, additional variant(s) that are in linkage disequilibrium with the variants and/or haplotypes of the present invention can be identified by a haplotyping method known in the art, as will be apparent to a skilled artisan in the field of genetics and haplotyping. The additional variants that are in linkage disequilibrium with a variant or haplotype of the present invention can also be useful in the various applications as described below.

For purposes of genotyping and haplotyping, both genomic DNA and mRNA/cDNA can be used, and both are herein referred to generically as “gene.”

Numerous techniques for detecting nucleotide variants are known in the art and can all be used for the method of this invention. The techniques can be protein-based or nucleic acid-based. In either case, the techniques used must be sufficiently sensitive so as to accurately detect the small nucleotide or amino acid variations. Very often, a probe is utilized which is labeled with a detectable marker. Unless otherwise specified in a particular technique described below, any suitable marker known in the art can be used, including but not limited to, radioactive isotopes, fluorescent compounds, biotin which is detectable using strepavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a nucleic acid-based detection method, target DNA sample, i.e., a sample containing genomic DNA, cDNA, and/or mRNA, corresponding to the one or more auxiliary genes must be obtained from the individual to be tested. Any tissue or cell sample containing the genomic DNA, mRNA, and/or cDNA (or a portion thereof) corresponding to the one or more auxiliary genes can be used. For this purpose, a tissue sample containing cell nucleus and thus genomic DNA can be obtained from the individual. Blood samples can also be useful except that only white blood cells and other lymphocytes have cell nucleus, while red blood cells are a nucleus and contain only mRNA. Nevertheless, mRNA is also useful as it can be analyzed for the presence of nucleotide variants in its sequence or serve as template for cDNA synthesis. The tissue or cell samples can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of a particular nucleotide variant, one technique is simply sequencing the target genomic DNA or cDNA, particularly the region encompassing the nucleotide variant locus to be detected. Various sequencing techniques are generally known and widely used in the art including the Sanger method and Gilbert chemical method. The newly developed pyrosequencing method monitors DNA synthesis in real time using a luminometric detection system. Pyrosequencing has been shown to be effective in analyzing genetic polymorphisms such as single-nucleotide polymorphisms and thus can also be used in the present invention. See Nordstrom et al., Biotechnol. Appl. Biochem., 31(2):107-112 (2000); Ahmadian et al., Anal. Biochem., 280:103-110 (2000).

Alternatively, the restriction fragment length polymorphism (RFLP) and AFLP method may also prove to be useful techniques. In particular, if a nucleotide variant in the target DNA corresponding to the one or more auxiliary genes results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998).

The presence or absence of a nucleotide variant at a particular locus in the one or more auxiliary genes of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA or mRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present invention is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in the one or more auxiliary genes, two oligonucleotides can be synthesized, one having the sequence just 5′ upstream from the locus with its 3′ end nucleotide being identical to the nucleotide in the variant locus of the particular auxiliary gene, the other having a nucleotide sequence matching the sequence immediately 3′ downstream from the locus in the auxiliary gene. The oligonucleotides can be labeled for the purpose of detection. Upon hybridizing to the target auxiliary gene under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to an auxiliary gene allele having a particular gene variant at a particular locus but not to other alleles can be designed by methods known in the art. The probes can have a length of, e.g., from 10 to about 50 nucleotide bases. The target auxiliary DNA and the oligonucleotide probe can be contacted with each other under conditions sufficiently stringent such that the nucleotide variant can be distinguished from the wild-type auxiliary gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in a RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art. See e.g., Roberts et al., Nucleic Acids Res., 25:3377-3378 (1997).

In the mutS assay, a probe can be prepared matching the auxiliary gene sequence surrounding the locus at which the presence or absence of a mutation is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques, and can all be useful in detecting mutations or nucleotide variants in the present invention. For example, the “sunrise probes” or “molecular beacons” utilize the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpin-shaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the auxiliary gene spanning the variant locus of interest and to differentially hybridize with different auxiliary alleles. The two ends of the probe are labeled with a quenching fluorophore and a reporter fluorophore, respectively. The TaqMan probe is incorporated into a PCR reaction for the amplification of a target gene region containing the locus of interest using Taq polymerase. As Taq polymerase exhibits 5′-3′ exonuclease activity but has no 3′-5′ exonuclease activity, if the TaqMan probe is annealed to the target DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present invention can also employ a chemiluminescence-based technique. For example, an oligonucleotide probe can be designed to hybridize to either the wild-type or a variant auxiliary gene locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in the auxiliary gene in accordance with the present invention can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Another useful technique that is gaining increased popularity is mass spectrometry. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present invention. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations. The microchip technologies combined with computerized analysis tools allow fast screening in a large scale. The adaptation of the microchip technologies to the present invention will be apparent to a person of skill in the art apprised of the present disclosure. See, e.g., U.S. Pat. No. 5,925,525 to Fodor et al; Wilgenbus et al., J. Mol. Med., 77:761-786 (1999); Graber et al., Curr. Opin. Biotechnol., 9:14-18 (1998); Hacia et al., Nat. Genet., 14:441-447 (1996); Shoemaker et al., Nat. Genet., 14:450-456 (1996); DeRisi et al., Nat. Genet., 14:457-460 (1996); Chee et al., Nat. Genet., 14:610-614 (1996); Lockhart et al., Nat. Genet., 14:675-680 (1996); Drobyshev et al., Gene, 188:45-52 (1997).

As is apparent from the above survey of the suitable detection techniques, it may or may not be necessary to amplify the target DNA, i.e., the gene, cDNA, mRNA, or a portion thereof to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2^{nd} ed., Cold Spring Harbor Laboratory, Cold Spring Harbor, N.Y., 1989. However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Collins et al., Nucleic Acids Res., 25:2979-2984 (1997); Horn et al., Nucleic Acids Res., 25:4835-4841 (1997); Horn et al., Nucleic Acids Res., 25:4842-4849 (1997); Nilsen et al., J. Theor. Biol., 187:273-284 (1997).

In yet another technique for detecting single nucleotide variations, the Invader® assay utilizes a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader® system utilizes two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, Sniper™, a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. See Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In SERRS, a chromophore-nucleic acid conjugate is absorbed onto colloidal silver and is irradiated with laser light at a resonant frequency of the chromophore. See Graham et al., Anal. Chem., 69:4703-4707 (1997). The fluorescence correlation spectroscopy is based on the spatio-temporal correlations among fluctuating light signals and trapping single molecules in an electric field. See Eigen et al., Proc. Natl. Acad. Sci. USA, 91:5740-5747 (1994). In single-molecule electrophoresis, the electrophoretic velocity of a fluorescently tagged nucleic acid is determined by measuring the time required for the molecule to travel a predetermined distance between two laser beams. See Castro et al., Anal. Chem., 67:3181-3186 (1995).

In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a nucleotide variant in the one or more auxiliary gene of a particular individual should be apparent to a skilled artisan apprised of this disclosure.

Protein-based detection techniques may also prove to be useful, especially when the nucleotide variant causes amino acid substitutions or deletions or insertions or frameshift that affect the protein primary, secondary or tertiary structure. To detect the amino acid variations, protein sequencing techniques may be used. For example, a protein or fragment thereof corresponding to an auxiliary gene can be synthesized by recombinant expression using an auxiliary DNA fragment isolated from an individual to be tested. Preferably, an auxiliary cDNA fragment of no more than 100 to 150 base pairs encompassing the polymorphic locus to be determined is used. The amino acid sequence of the peptide can then be determined by conventional protein sequencing methods. Alternatively, the recently developed HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected therefrom is analyzed. See Gatlin et al., Anal. Chem., 72:757-763 (2000).

Other useful protein-based detection techniques include immunoaffinity assays based on antibodies selectively immunoreactive with mutant auxiliary gene encoded protein according to the present invention. The method for producing such antibodies is described above in detail. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well-known antibody-based techniques can also be used including, e.g., enzyme-linked immunosorbent assay (ELISA), radioimmunoassay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530, both of which are incorporated herein by reference.

Accordingly, the presence or absence of one or more auxiliary genes nucleotide variant or amino acid variant in an individual can be determined using any of the detection methods described above.

Typically, once the presence or absence of one or more auxiliary gene nucleotide variants or amino acid variants is determined (or the status of the biomarkers in Table 1), physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of an auxiliary nucleotide variant of the present invention in the individual tested can be embodied in descriptive statements, diagrams, photographs, charts, images or any other visual forms. For example, images of gel electrophoresis of PCR products can be used in explaining the results. Diagrams showing where a variant occurs in an individual's auxiliary gene are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a nucleotide variant or amino acid variant in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present invention also encompasses a method for producing a transmittable form of information on the genotype of the two or more suspected cancer samples from an individual. The method comprises the steps of (1) determining the genotype of the DNA from the samples according to methods of the present invention; and (2) embodying the result of the determining step in a transmittable form. The transmittable form is the product of the production method.

### Kits

The present invention also provides a kit for genotyping the one or more auxiliary genes, i.e., determining the presence or absence of one or more of the nucleotide or amino acid variants in one or more auxiliary genes in a sample obtained from a patient. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting nucleotide or amino acid variants discovered in accordance with the present invention using the above-discussed detection techniques.

The kits of the invention can include the probes and reagents described above for detecting the one or more biomarkers of the invention, and optionally include reagents and probes for analyzing one or more auxiliary genes, or for re-analysis of one or more of the biomarkers of the invention.

In one embodiment, the detection kit includes one or more oligonucleotides useful in detecting one or more of the nucleotide variants in one or more auxiliary genes. Preferably, the oligonucleotides are allele-specific, i.e., are designed such that they hybridize only to a mutant auxiliary gene containing a particular nucleotide variant discovered in accordance with the present invention, under stringent conditions. Thus, the oligonucleotides can be used in mutation-detecting techniques such as allele-specific oligonucleotides (ASO), allele-specific PCR, TaqMan, chemiluminescence-based techniques, molecular beacons, and improvements or derivatives thereof, e.g., microchip technologies. The oligonucleotides in this embodiment preferably have a nucleotide sequence that matches a nucleotide sequence of a variant auxiliary gene allele containing a nucleotide variant to be detected. The length of the oligonucleotides in accordance with this embodiment of the invention can vary depending on its nucleotide sequence and the hybridization conditions employed in the detection procedure. Preferably, the oligonucleotides contain from about 10 nucleotides to about 100 nucleotides, more preferably from about 15 to about 75 nucleotides, e.g., contiguous span of 18, 19, 20, 21, 22, 23, 24 or 25 to 21, 22, 23, 24, 26, 27, 28, 29 or 30 nucleotide residues of a an auxiliary gene nucleic acid. Under most conditions, a length of 18 to 30 may be optimum. In any event, the oligonucleotides should be designed such that it can be used in distinguishing one nucleotide variant from another at a particular locus under predetermined stringent hybridization conditions. Preferably, a nucleotide variant is located at the center or within one (1) nucleotide of the center of the oligonucleotides, or at the 3′ or 5′ end of the oligonucleotides. The hybridization of an oligonucleotide with a nucleic acid and the optimization of the length and hybridization conditions should be apparent to a person of skill in the art. See generally, Sambrook et al., Molecular Cloning: A Laboratory Manual, 2^{nd} ed., Cold Spring Harbor Laboratory, Cold Spring Harbor, N.Y., 1989. Notably, the oligonucleotides in accordance with this embodiment are also useful in mismatch-based detection techniques described above, such as electrophoretic mobility shift assay, RNase protection assay, mutS assay, etc.

In another embodiment of this invention, the kit includes one or more oligonucleotides suitable for use in detecting techniques such as ARMS, oligonucleotide ligation assay (OLA), and the like. The oligonucleotides in this embodiment include an auxiliary gene sequence of about 10 to about 100 nucleotides, preferably from about 15 to about 75 nucleotides, e.g., contiguous span of 18, 19, 20, 21, 22, 23, 24 or 25 to 21, 22, 23, 24, 26, 27, 28, 29 or 30 nucleotide residues immediately 5′ upstream from the nucleotide variant to be analyzed. The 3′ end nucleotide in such oligonucleotides is a nucleotide variant in accordance with this invention.

The oligonucleotides in the detection kit can be labeled with any suitable detection marker including but not limited to, radioactive isotopes, fluorophores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with certain proteins or polypeptides (encoded by the auxiliary genes) containing specific amino acid variants discovered in the present invention. Methods for producing and using such antibodies have been described above in detail.

Various other components useful in the detection techniques may also be included in the detection kit of this invention. Examples of such components include, but are not limited to, Taq polymerase, deoxyribonucleotides, dideoxyribonucleotides other primers suitable for the amplification of a target DNA sequence, RNase A, mutS protein, and the like. In addition, the detection kit preferably includes instructions on using the kit for detecting nucleotide variants in auxiliary gene sequences.

### Therapeutic Agents

In some aspects, the methods, biomarkers, and compositions of the invention are useful for selecting a therapeutic treatment for a patient having a particular biomarker profile. According to these embodiments, the set of biomarkers is used to select a treatment for a cancer based on the association of a biomarker signature with response or lack of response to a particular therapeutic or class of therapeutics. In one aspect of the invention, the methods and biomarkers are used to classify patients as responders and non-responders to a particular therapeutic.

In one aspect of the invention, the therapeutic is an antibody to EGFR, HER2, HER3, and/or HER4.

In one aspect of the invention, the therapeutic is small molecule targeting EGFR, HER2, HER3, and/or HER4.

In one aspect of the invention, the therapeutic is an antibody to EGFR. In one embodiment the antibody to EGFR is chosen from cetuximab, panitumumab, nimotuzumab, and matuzumab.

In one aspect of the invention, the therapeutic is an antibody to HER2 In one embodiment, the antibody to HER2 is chosen from trastuzumab and pertuzamab.

In one aspect of the invention, the therapeutic is an antibody to VEGF. In one embodiment the antibody to VEGF is chosen from bevacizumab and ranibizumab.

In one aspect of the invention, the therapeutic is a small molecule EGFR inhibitor. In one aspect of the invention, the small molecule EGFR inhibitor is chosen from gefitinib and erlotinib.

In one aspect of the invention, the therapeutic is a small molecule EGFR/HER2 inhibitor. In one aspect of the invention, the small molecule EGFR/HER2 inhibitor is chosen from lapatinib (tykerb;gw572016), zd6474 (zactima), hki-272 (wyeth), BIBW-2992, AEE788 (Novartis), BMS-599626, x1-647 (Exelixis).

In one aspect of the invention, the therapeutic is a small molecule ErbB inhibitor. In one aspect of the invention, the small molecule ErbB inhibitor is CI-1033 (Pfizer; PD183805)

In one aspect of the invention, the therapeutic is a small molecule AKT inhibitor. In one aspect of the invention, the small molecule AKT inhibitor is chosen from Deguelin and perifosine.

In one aspect of the invention, the therapeutic is a small molecule PIK3CA inhibitor. In one aspect of the invention, the small molecule PIK3CA inhibitor is PX-866.

In one aspect of the invention, the therapeutic is a small molecule mTOR inhibitor. In one aspect of the invention, the small molecule mTOR inhibitor is chosen from LY294002 (rapamycin), CCI-779 (Temsirolimus), Everolimus (RAD001), and AP23573.

In one aspect of the invention, the therapeutic is a small molecule inhibitor of a target downstream of PTEN.

In one aspect of the invention, the therapeutic is a small molecule inhibitor of MEK. In one aspect of the invention, the small molecule inhibitor of MEK is azd6244

In one aspect of the invention, the therapeutic is a small molecule prenylation inhibitor. In one aspect of the invention, the small molecule prenylatrion inhibitor is chosen from azd3409, 4-(2-(4-(8-chloro-3,10-dibromo-6,11-dihydro-5H-benzo-(5,6)-cyclohepta(1,2-b)-pyridin-11(R)-yl)-1-piperidinyl)-2-oxo-ethyl)-1-piperidinecarboxamide (SCH66336), and methyl {N-[2-phenyl-4-N[2(R)-amino-3-mercaptopropylamino]benzoyl]}-methionate (FTI-277).

In one aspect of the invention, the therapeutic is a small molecule Src inhibitor. In one aspect of the invention, the small molecule src inhibitor is azd0530

In one aspect of the invention, the therapeutic is an IGF1R inhibitor or antibody. In one aspect of the invention, the IGF1R inhibitor or antibody is MAb cp-751871.

In one aspect of the invention, the therapeutic is a small molecule IGF1R kinase inhibitor. In one aspect of the invention, the small molecule IGF1R kinase inhibitor is NVP-AEW541.

In one aspect of the invention, the methods and biomarkers are used to classify patients as responders and non-responders to trastuzumab. In a related aspect, the methods and biomarkers are used to classify patients as responders and non-responders to lapatinib. In another related aspects the methods and biomarkers of the invention are used to aid in determining whether a patient should be treated with trastuzumab and/or lapatinib.

### EXAMPLES

The following examples are included to demonstrate preferred embodiments of the invention. It should be appreciated by those of skilled the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

A set of genes useful for characterizing a cancer or tissue is shown Table 1, above. These genes can be assayed for mutations (by resequencing nucleic acids from tumor or cancer samples), RNA expression levels (e.g., by real-time RT-PCR), DNA copy number analysis, and/or protein expression analysis. In some specific aspects of the invention, DNA and/or RNA can be isolated from FFPE (formalin-fixed paraffin embedded) tumor samples. The status of one or more biomarkers from Table 1 can be correlated with response to trastuzamab or lapatinib, or other therapeutic treatment.

Probes useful for detecting the biomarkers in Table 1 are commercially available and/or described in the literature. For example, monoclonal and polyclonal antibodies are commercially available that specifically bind many of the protein biomarkers in Table 1. In addition, reagents and protocols for RT-PCR and quantitative PCR for many of the biomarkers are commercially available and/or published.

A retrospective set of breast cancer samples can be used in this analysis. The samples can be from patients with metastatic disease, who have been treated with Herceptin, and have known clinical outcomes. Prior to any molecular analysis, the tumor samples can be classified as responders or non-responders (according to diagnostic protocol). 150 patients (equally divided between responders and non-responders) can be profiled for the genes and/or expression products in Table 1. In order to be clinically relevant, the test must have a high negative predictive value (0.95, chance that a predicted non-responder will not respond) and a reasonable frequency of negative predictions (0.20). The high negative predictive value insures that patients are not wrongly directed away from Herceptin, and the modest frequency of negative predictions insures that a reasonable fraction of test results alter clinical practice.

The molecular data can be correlated to clinical outcome considering multiple biomarkers. To avoid a combinatorial analysis that can create a significant multiple testing problem, initially, each marker can be considered separately. With a test having about 50 assays, a p-value of, e.g., 0.001 can be required before considering any marker to be associated with clinical outcome (false discovery rate of 0.05).

After a mutation or expression profile is associated with response, it can be a candidate for inclusion in the diagnostic test. The associated-profiles can be combined in various ways in an attempt to define the most predictive algorithm. The associated multiple testing problem can be adjusted for by analyzing simulations with permutated clinical outcomes, and/or other statistical techniques as deemed necessary.

After development, the predictive algorithm is validated on a naïve set of tumor samples in blinded fashion. In this case, there is no multiple testing, so the required sample size can be calculated using p-value of 0.05 and fixing other parameters as before (Table 3). The validation cohort should contain at least 80 subjects (Table 4).

Paraffin-embedded tissue samples from breast cancer patients treated with trastuzumab-based therapy between Sep. 1, 1998, and March 2006 will be can be analyzed. Each representative tumor block can be characterized by standard histopathology for diagnosis, semi-quantitative assessment of amount of tumor, and tumor grade. A total of 3 sections (5 microns thickness each) can be prepared and placed in 2 Costar tubes (3 sections in each tube) for all cases.

The tissue information can be traced back to clinical information for clinical-biological correlations. Medical records can be reviewed to retrospectively evaluate the disease outcomes associated with trastuzumab when used alone or in combination with other antitumor agents in metastatic breast cancer patients. The data that can be retrieved include, but are not limited to patient demographics, cancer stage, tumor characteristics, prior and concurrent anticancer therapies, dosing and administration details related to trastuzumab and concurrent chemotherapy, duration of therapy, and recurrence and survival information.

Correlations can be made between molecular markers listed on Table 1 and clinical outcome, time to progression and overall survival. Overall survival can be determined from the date of the first infusion (start-date) to the time of death. Patients who are still alive at the end of follow-up can be censored from this analysis. Time to progression can be determined from the date of the first infusion (start-date) until disease progression has been documented in the medical record (physician note). Because trastuzumab may be continued beyond progression, duration of therapy with trastuzumab can also be determined (start-date to date of final infusion). The information gathered can be used as part of a larger statistical analysis.

The sample consists of patients who have been treated with Herceptin, who can be classified as responders or non-responders, and as positive or negative for each biomarker. Individuals who do not carry the biomarker are predicted to be non-responders, so the biomarker is a negative predictor of response. The negative predictive power (NP) is the probability that non-carriers of the biomarker will be non-responders.

TABLE 2
Example

| | Biomarker+ | Biomarker− | |
|---|---|---|---|
| Responders | 95 | 5 | 100 |
| Non-responders | 115 | 85 | 200 |
| | 210 | 90 | 300 |

total sample size = 300
responder frequency = 100/300 = 0.33
negative predictor frequency = 90/300 = 0.30
NP = negative predictive power = 85/90 = 0.95
Chi-Square test p-value = 6e−11
Sample size estimates

Sample size estimates are calculated for Pearson's Chi-Square test of the contingency table assuming 80% power. Alpha is set at 0.001 for the discovery sample, which will require multiple testing to select candidate biomarkers, and 0.05 for the confirmation sample, which will be used to test the predictive algorithm. For each sample size calculation, the responder frequency is assumed to be ⅓, and two other parameters are fixed: the negative predictor frequency and the negative predictive power (NP). Together with the total sample size, these parameters determine the cell counts in the table. Sample size calculations are made for a test comparing the proportion of negative predictors in responders versus non-responders (S-PLUS v 7.0.3 for Linux 2005, Insightful Corp. Seattle).

### Tables 3 & 4

TABLE 3
Sample sizes for
discovery

| | Negative predictive power vs | | | |
|---|---|---|---|---|
| 80% power alpha 0.001 | Negative predictor frequency | | | |
| Responders:Non-Responders 1:1 | 0.90 | 0.95 | 0.98 | 0.99 |
| 0.1 | 504 | 334 | 268 | 250 |
| 0.2 | 230 | 152 | 122 | 114 |
| 0.3 | 138 | 92 | 74 | 70 |
| 0.4 | 92 | 62 | 50 | 46 |
| 0.5 | 64 | 44 | 36 | 32 |
| 0.6 | 46 | 32 | 26 | 24 |

TABLE 4
Sample size for
validation

| | Negative predictive power vs | | | |
|---|---|---|---|---|
| 80% power alpha 0.05 | Negative predictor frequency | | | |
| Responders:Non-Responders 1:1 | 0.90 | 0.95 | 0.98 | 0.99 |
| 0.1 | 252 | 170 | 138 | 130 |
| 0.2 | 116 | 78 | 64 | 60 |
| 0.3 | 70 | 48 | 40 | 36 |
| 0.4 | 48 | 32 | 26 | 24 |
| 0.5 | 34 | 24 | 18 | 18 |
| 0.6 | 24 | 16 | 14 | 12 |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing invention has been described in some detail by way of illustration and example for purposes of clarity of understanding, it will be obvious that certain changes and modifications may be practiced within the scope of the appended claims.

What is claimed is:

1. A method for selecting a therapeutic treatment for a breast cancer patient, said method comprising:
measuring the level at least 4, 6, 7, 8, 9, 10, 11, 12, 13, 14, or 15 or more biomarkers in Table 1, from a tumor, tissue, or cell sample from a patient;
correlating the levels of the biomarkers to the biomarker profile for response to trastuzumab or lapatinib;
and selecting a therapeutic treatment based on the comparison of the biomarker profile from said tumor or tissue sample and the biomarker profile for response to trastuzumab or lapatinib.
